(19) United States
(12) Patent Application Publication
(10) Pub. No.: US 2024/0174977 A1
(43) Pub. Date: May 30, 2024

(54) METHODS FOR IMPROVING EARLY EMBRYO DEVELOPMENT

(71) Applicant: SHANDONG UNIVERSITY, Jinan, Shandong (CN)

(72) Inventors: Zijiang CHEN, Jinan, Shandong (CN); Han ZHAO, Jinan, Shandong (CN); Keliang WU, Jinan, Shandong (CN); Honghui ZHANG, Jinan, Shandong (CN)

(21) Appl. No.: 18/279,936

(22) PCT Filed: Apr. 27, 2022
(86) PCT No.: PCT/CN2022/089671

§ 371 Date: Sep. 1, 2023

Related U.S. Application Data
(60) Provisional application No. 63/180,926, filed on Apr. 28, 2021.

(51) Int. Cl. C12N 5/073

(52) U.S. Cl. CPC C12N 5/0604; C12N 2501/727

(57) ABSTRACT

The general inventive concepts are based on the discovery that mammalian fertility and embryo development can be enhanced by a CHK1 inhibitor. A method for treating human infertility is disclosed. The method comprises identification of an individual having altered CHK1 function and administering a therapeutically effective amount of a CHK1 inhibitor. Further, embryonic development can be enhanced by the use of a culture medium comprising CHK1 inhibitor.

### CROSS-REFERENCE TO RELATED APPLICATIONS

This application is the claims priority to and the benefit of U.S. Provisional Patent Application No. 63/180,926, filed Apr. 28, 2021, the entire contents of which are incorporated by reference as if fully recited herein.

### REFERENCE TO SEQUENCE LISTING

The content of the ASCII text file of the sequence listing named 36682.04011_ST25.txt which is 8 kb in size was created on Apr. 22, 2022, and filed concurrently herewith, is hereby incorporated by reference in its entirety.

### FIELD OF DISCLOSURE

This disclosure relates generally to methods of treating infertility, particularly infertility that is related to zygote arrest and/or embryonic development.

### BACKGROUND

Reproductive health is crucial to maintaining population sustainability, however, with the continuous changes in the natural and social environment in which human beings live, issues surrounding fertility and fertility rate are causing concern. The global prevalence of infertility increased from 11.0% in 1997 to 16.4% in 2019 and is expected to rise to 17.2% by 2023. Affected by factors such as environmental pollution, delayed childbearing age, and life pressure, the number of infertile people is still increasing. In order to solve the reproductive dilemma of infertile individuals, assisted reproductive technology (ART) came into being and developed rapidly, and has been widely used in the world. In some low-fertility countries in northern Europe, 7% of births are born through ART each year. In recent years, the amount of in vitro fertilization (IVF) and intracytoplasmic sperm injection (ICSI) treatment in a number of countries. However, the successful pregnancy rate of assisted reproductive technology is still low at present, and it is urgent for researchers to further improve the embryo culture system, especially the embryo culture system from in vitro fertilization to transplantation. It is a feasible and effective method to add a suitable amount of factors that can promote embryonic development to the existing commonly used embryo culture medium to aid in many stages of embryo development.

Mammalian egg fertilization is a complex multi-stage process. Fertilization features the transformation of two highly specialized meiotic germ cells, the oocyte and the sperm, into a totipotent zygote. This transformation triggers a complex cellular program that likely represents the most intricate cell transition in mammalian/human biology. Failure in any of the requisite steps of the process described can lead to infertility.

ART, including in IVF) and ICSI, enable infertile women to have their biological embryos in vitro and further give birth to babies after embryo transfer. It has been estimated that about 10% of all human embryos produced by ART were blocked in the very early embryo stage and approximately 2% fertilized oocytes derived from ART could not accomplish the first cell division. About one half of human infertility cases involve an underlying genetic factor, although the majority of genetic causes have remained elusive. One substantial cause of unsuccessful development of a fertilized egg is zygote arrest (ZA). The genetic determinants and suitable clinical treatment of female infertility caused by zygote arrest remain largely unknown.

### SUMMARY

There is an unmet need for compositions and methods that treat, prevent, or otherwise ameliorate the symptoms associated with mammalian zygote arrest and/or infertility.

The general inventive concepts are based, in part, on the recognition that enhanced kinase activity can block embryonic development, more particularly, that increased CHK1 expression and/or exposure plays a role in mammalian (in)fertility and embryonic development. This is based on the discovery that CHK1 mutations show increased kinase activity and the application of a CHK1 inhibitor was able to significantly rescue the phenotype in both mouse and human, effectively reversing/treating zygote arrest. Further, the general inventive concepts recognize that exposing an embryo to a certain concentration of a CHK1 inhibitor (e.g., in a culture medium) can enhance (e.g., accelerate) embryonic development while avoiding issues associated with embryo quality.

The general inventive concepts recognize a method for the treatment of infertility comprising, identifying an individual having altered CHK1 function, and administering therapeutically effective amount of a CHK1 inhibitor.

The general inventive concepts also relate to a culture medium for mammalian embryo culturing, the culture medium comprising a therapeutically effective amount of a CHK1 inhibitor. In certain embodiments, the additive is a CHK1 inhibitor in an amount of 0.1 nM to 100 nM.

The general inventive concepts also relate to and contemplate a method for enhancing embryonic development. The method comprises providing a culture medium, adding a therapeutically effective amount of a CHK1 inhibitor and contacting the embryo with the culture medium. In certain exemplary embodiments, embryonic development is enhanced by modulating blastocyst development rate.

The general inventive concepts also relate to and contemplate a method for the treatment and/or prevention of zygote arrest, the method comprises identifying a subject suffering from zygote arrest or at increased risk of zygote arrest, contacting a zygote from the individual with a therapeutically effective amount of a CHK1 inhibitor. In certain exemplary embodiments, the therapeutically effective amount corresponds to an amount sufficient to overcome the zygote arrest.

The general inventive concepts also relate to and contemplate a composition for the treatment and/or prevention of zygote arrest, the composition comprises a therapeutically effective amount of a CHK1 inhibitor.

The general inventive concepts also relate to and contemplate method for treating altered kinase activity, the method comprising identifying an individual having altered CHK1 function, and administering therapeutically effective amount of a CHK1 inhibitor. In certain embodiments the individual has increased kinase activity resulting in zygote arrest and/or infertility.

### BRIEF DESCRIPTION OF DRAWINGS

FIG. 1 shows a western blotting and bar graphs of the expression pattern of CHK1 in oocytes and pre-implantation embryos. FIG. 1A: Western blot result showed the expression of CHK1 in mouse oocytes and early embryos at different stages. GAPDH was used as a loading control. FIG. 1B shows the relative quantitative result of the protein expression of CHK1 in mouse oocytes and early embryos. FIG. 1C: RT-PCR results show the mRNA level of CHK1 in mouse oocytes and early embryos. FIG. 1D The RNA-seq result of CHK1 in human mature oocytes and preimlpantation embryos. Bars indicate means t SEM.

FIG. 2 illustrates CHK1 mutations lead to mouse zygote arrest. FIG. 2A shows a diagram of mouse zygote injection flow MII oocytes and capacitated spermatozoa were fertilized in vitro for 4 hours, following that wild-type or mutant hCHK1 cRNA, which were EGFP tagged, was injected into the cytoplasm of fertilized eggs and subsequently cultured for 18 hours to observe the zygote cleavage rate. FIG. 2B shows images of mouse zygotes overexpressing wild type or mutant human EGFP-CHK1 after 18 hours. Scale bar:100 um.

FIG. 3 illustrates conservation analysis of the mutated amino acid residues and structural exhibition of CHK1. FIG. 3A shows sequence alignment reveals evolutionary conservation of amino acid residues R379 in Family 1, F441 in Family 2, R442 in Family 3 and R420 in Family 4 in nine species. FIG. 3B shows an overview of the predicted structure of the wild-type CHK1 protein (Left). Arrow indicates the location of R379, R442 and R420. Magnified view of the predicted structures surrounding R379 (a) and R442 (c) and their altered structures after mutation (b and d) (Right). The yellow dashed lines indicated by black arrowheads represent the predicted hydrogen bonds. The arrow marks the mutated amino acid Q442 and the amino acid L443 with newly formed hydrogen bond.

FIG. 4 is a schematic representation showing that the R379Q mutation and R442Q mutation converted the positively charged patch to the negatively charged patch (in yellow circle) on the surface of CHK1.

FIG. 5 illustrates the changed nuclear-cytoplasm localization of CHK1 proteins is due to the mutations in NES or NLS. HEK-293 cells were treated with DMSO or leptomycin B (LMB), a Crm1 inhibitor to inhibit nuclear export signal, for 15 h after transfection for 30 h. Red: mCherry-WT CHK1, Green: EGFP-WT or -mutated CHK1, Blue:DAPI, Scale bar:10 um. (A) It shows that the mutant groups had a tendency of cytoplasmic localization compared with wild-type group, especially the mutation F441fs*16 in DMSO treatment group. (B) LMB treatment groups exhibit almost completely nuclear localization, except F441fs*16.

FIG. 6A is a diagram indicating the auto-inhibitory regulation of CHK1. Under normal growth condition, kinase domain in N-terminal of CHK1 interacts with the C-terminal regulatory domain to form a “closed” structure maintaining inactive form (upper). In the presence of DNA damage factors, the phosphorylation of CHK1 will break the interaction between N-terminal and C-terminal and expose the kinase domain, inducing the activation of CHK1 (lower). Blue boxes: the two conserved motifs in C-terminal, CM1 and CM2. FIG. 6B shows HEK-293T cells were collected to assay kinase activity 48 hours after transfection of wild-type or mutant CHK1 plasmids, followed by the treatment of 500 nM CPT for another 2 hours. The results showed that the relative kinase activity (OD_{wt or mutant group}/OD_{wt group} in each replicate) of each mutation group was higher than that of the wild-type group, though the R420K mutation showed no significant difference (t-test). Bars indicate means f SEM, ns: no significant difference.

FIG. 7A shows HEK-293T cells were separately treated with DMSO or PF477736 (150 nM) for another 18 hours after transfection for 30 hours. The western blot results indicated that CHK1 inhibitor PF477736 could significantly reduce the expression of CHK1 downstream proteins (pCDC25C and pCDK1s). FIG. 7B is a bar graph showing the results when Mouse zygotes injected with wild-type or mutant CHK1 cRNAs were treated with DMSO or PF477736 (10 nM), respectively. 18 hours later, the cleavage results in each group were documented here, indicating that the inhibitor could significantly increase the mitosis rate of zygotes carrying the mutations (p<0.05). The Chi-square test was used and a total of about 90 zygotes were calculated in each group in three repeats. FIG. 7C is a bar graph showing the blastocyst rate of zygotes carrying wild-type or mutant hCHK1 under DMSO or different PF477736 concentrations (1/10/100 nM).

FIG. 8 shows images of mouse zygotes injected with wild-type or mutant CHK1 cRNAs were treated with DMSO or PF477736 (10 uM), respectively.

FIG. 9 shows CNV-seq results for blastocysts derived from control zygotes or zygotes overexpressing mutant CHK1. The genome sequence of mutant blastocysts was aligned with the sequence of normal control blastocysts. The results pointed out that there were no chromosome aneuploidy abnormalities or chromosomal deletions or duplications larger than 4 Mb in mutant blastocysts treated with PF477736.

FIG. 10 is a bar graph showing the measured weight of pups per group five weeks after birth and there was no significant difference between the wild-type (WT) or mutated (F441fs*16/R379Q) groups treated by PF477736 and the normal control group (t-test). Bars indicate means f SEM, ns: no significant difference.

FIG. 11 is a diagram showing the activated role of CHK1 in zygote arrest. The four mutations we identified are located in two highly conserved regions in the C-terminal of CHK1 (CM1 and CM2), which are relating to the auto-inhibitory regulation of CHK1. Our results demonstrate that those mutations have increased activity though without the stimulation of DNA damage signal. The mutations may expose their kinase domain by altering the protein conformation resulting in activation in normal conditions, and produce more inhibitory phosphorylated CDC25C/CDK1. It has been previously reported that inhibition of CDK1 seriously inhibited migration and fusion of male and female pronuclei in starfish fertilized eggs. The accumulation of inhibitory pCDK1s may disturbance the fusion of male and female pronuclei and render cell cycle arrest in human zygotes.

FIG. 12 shows the pedigrees of four families with inherited or de novo CHK1 mutations. All affected individuals presented a single allele mutation but carrier men did not suffer from the disease, which is characterised by female-limited autosomal dominant inheritance. The CHK1 mutation c. 1136G>A in Family 1(III-1 and III-2) is inherited from their father while the mutations c.1323delC in Family 2 and 1325G>A in Family 3 are de novo, as the parents were not carriers. The squares denote a male family member, circles female family member, solid symbols affected subjects, open symbols unaffected ones. Slashes indicate death, question marks represent unknown fertility status, and the arrows mark the probands in Families 1 and 4. The CHK1 genotypes are marked below the corresponding family members, and “W” represents wild type. The Sanger sequencing chromatograms are shown below the pedigrees.

FIG. 13A is a bar graph showing the zygote cleavage rate, shown as the overall rate across three experiments (about 90 eggs in each group), was significantly decreased in mouse zygotes with mutated RNA compared with wild-type eggs (P<0.05), based on the chi-square test. FIG. 13B shows immunofluorescence results of mouse zygotes with mutations or the 2-cell stage embryo with wild-type CHK1. The fertilized eggs of mice were injected with either wild-type or mutant hCHK1 cRNAs and then cultured in vitro for 18 hours to be fixed for immunofluorescence. Green:EGFP-tagged wild-type or mutant CHK1, Blue:DAPI, Scale bar:10 um. FIG. 13C is a schematic diagram of the CHK1 protein showing its kinase domain, the C-terminal domain with SQ, CM1 and CM2 motifs, and the location of altered amino acids. NES: nuclear export signal; NLS: nuclear localization signal. FIG. 13D shows the immunofluorescence results of HEK-293 cells co-transfected with mCherry-WT CHK1 and EGFP-CHK1 (WT or mutant) to show the intracellular localization of proteins. Red: mCherry-WT CHK1; Green:EGFP-WT or EGFP-mutated CHK1; Blue:DAPI; Scale bar:10 um. FIG. 13E shows the relative intensity of nucleui compared to the total cell of wild-type or mutant CHK1 in HEK-293 cells. Error bars, S.E.M. ****P<0.0001 using two-tailed Student's t-tests.

FIG. 14A shows western blot analysis of HEK-293T cell extracts. HEK-293T cells were transfected with either EGFP-WT or mutant CHK1 constructs for 48 hours in order to assay downstream CHK1 proteins. The EGFP-WT group treated by 500 nM camptothecin (CPT, Sigma, C9911) to induce DNA damage serves as a positive control. FIG. 14B is a diagram showing the downstream pathway of CHK1 after activation. The activated CHK1 is able to phosphorylate CDC25C at S216 and thus lead to the accumulation of inhibitory phosphorylated CDK1 (at T14 and Y15), which blocked the G2-M transition. FIG. 14C shows mutations in the key phosphorylation sites CDC25C(S216) and CDK1(T14 and Y15) can ameliorate the zygote block phenotype in mouse zygotes. The residues in CDC25C(S216) and CDK1(T14 and Y15) were first mutated to alanine and then respectively overexpressed in mouse fertilized eggs together with the mutation F441fs*16. Representative images are shown. WT: wild-type, MT: mutant; Scale bar:100 uM. FIG. 14D is a bar graph showing the cleavage rate was significantly increased in zygotes carrying the mutated CDC25C and CDK1 (p<0.05; chi-square test). The total cleavage rates of three replicates are shown above the column. Approximately 80 eggs were used in each group.

FIG. 15A shows the blocked zygotes of patient III-2 (Family 1, p.R379Q) could resume cleavage with PF477736. The donated zygotes of the patient had been cultured until the third embryo day without cleavage and then undergone cryopreservation for further research. Applicants cultured those zygotes with PF477736 (10 nM) for another day after thawing to investigate the efficacy of the drug, and found that blocked zygotes were able to divide when treated with PF477736. On the contrary, zygotes treated with DMSO were not able to divide. FIG. 15B shows mouse zygotes overexpressing either wild-type or mutant CHK1 (p.F441fs*16 or p.R379Q) were cultured with or without PF477736(10 nM) until the 2-cell embryo stage, and then transferred to a new medium without DMSO or PF477736 in order to gain blastocysts. The representative images of blastocysts are shown. FIG. 15C is a bar graph showing the inhibitor markedly increased blastocyst rates in mutated groups (based on an unpaired t-test). Bars: SEM, ns: no significant difference. FIG. 15D is a bar graph showing the 2-cell embryos in the control group and in WT or mutated groups with PF477736 were transferred to pseudo-pregnant female mice in order to observe the litters (Table 3). There are considerable differences in birth rates between the normal control group without any treatment and WT or mutant groups treated with 10 nM PF477736 (p.F441fs*16 or p.R379Q) based on a t-test. Bars: SEM. FIG. 15E is representative image of the offspring (yellow dotted circle) in a mutant group (R379Q) is shown. FIG. 15F is a diagram depicting the flow of blastocyst culture at different concentrations and embryo transfer. The mouse zygotes overexpressing the mutation p.F441fs*16 or p.R379Q were treated with PF477736 at 1 nM, 10 nM or 100 nM until the 2-cell embryo stage, and the embryos were then transferred to a medium without PF477736 for culturing until the blastocyst stage; the mouse 2-cell embryos expressing WT CHK1 and mutation p.F441fs*16 or p.R379Q with PF477736 at 10 nM were transferred to pseudo-pregnant female mice in order to observe the litter.

FIG. 16A shows time-lapse imaging showing development progress of the embryo in control group and PF477736 treatment group (PF-1). FIG. 16B shows chromatograms of Sanger sequence of embryo PF-1 and PF-3. “W” represents wild-type, “M” represents mutant. FIG. 16C shows expression of human ESC markers in the two embryo stem cell lines derived from PF-1 and PF-3, including OCT4, SOX2, SSEA4, TRA-1-60 and TRA-1-81.

FIG. 17 shows the results of CNV-seq of the two embryonic stem cell lines derived from patient (III-2, Family 1). The two blastocysts, derived from the patient under the treatment with PF477736, were employed to establish embryonic stem cells, ESC_PF-1 (a) and ESC_PF-3 (b).

FIG. 18A is a schematic showing a general procedure for enhancing the development of a mammalian blastocyst.

FIG. 18B shows images of zygotes exposed to various concentrations of additive in a culture medium.

FIG. 18C is a graph showing the results of blastocyst development rate for zygotes exposed to various concentrations of additive in a culture medium.

FIG. 19A is a bar graph showing the results of qualitative assessment of embryos by grade.

FIG. 19B is an image showing immunofluorescence staining of the DNA damage marker protein γH2AX for embryos subjected to the medium dose of additive.

FIG. 19C shows CNV—seq analysis for embryos subjected to the medium dose of additive, and the results showed that the embryos in the inhibitor group were ploidy intact, and there was no obvious deletion or duplication of large chromatin fragments.

### DETAILED DESCRIPTION

Several illustrative embodiments will be described in detail with the understanding that the present disclosure merely exemplifies the general inventive concepts. Embodiments encompassing the general inventive concepts may take various forms and the general inventive concepts are not intended to be limited to the specific embodiments described herein.

Mammalian fertilization features the transformation of two highly specialized meiotic germ cells, the oocyte and the sperm, into a totipotent zygote. This transformation triggers a complex cellular program that likely represents the most intricate cell transition in human biology. The mature oocyte initially fuses with capacitated sperm to respectively form the female and male pronucleus, initiating the development of a new life. Subsequently, the two haploid pronuclei migrate and congregate to each other forming a two-cell embryo after the first symmetrical cleavage, which is a crucial transition from a successfully accomplished meiosis to beginning mitosis. After this, the two-cell embryo develops into a blastocyst after several consecutive mitotic events and differentiation. Failure in any of the steps of the process described above can cause human infertility. It was estimated that about 10% of all human embryos produced by assisted reproduction techniques (ART) were blocked in the very early embryo stage. WEE2 deficiency has been reported to result in human infertility characterized by a failure in the formation of the pronucleus. However, little is known about the genetic factors predominantly regulating female and male pronuclei fusion and the transition from meiosis to mitosis after fertilization.

Evolutionarily highly conserved DNA damage response and cell cycle checkpoint ensure genomic stability, in which the central is cell cycle checkpoint kinase 1 (CHK1). CHK1, a serine/threonine protein kinase that regulates the transition between the G2 and M phases of the cell cycle, was first identified in 1993 in fission yeast. This protein is of vital importance in genome maintenance, cancer therapy and early embryo development. Although CHK1 plays a critical role in mouse early embryonic development, the mechanisms behind the association of CHK1 in human pronuclei fusion and the initiation of the embryo mitosis are not well known.

The terms “susceptible” and “at risk” as used herein, unless otherwise specified, mean being genetically predisposed, having a family history of, and/or having symptoms of the condition or disease (e.g., showing unwanted expression of a marker or protein). The term refers to those having a vulnerability higher than the general population.

The terms “modulating” or “modulation” or “modulate” as used herein, unless otherwise specified, refer to the targeted movement of a selected characteristic (e.g., an expression level or symptom). In certain embodiments, the term refers to balancing or “right sizing” or “shaping” a biological response or expression level to a level akin to that of an otherwise healthy population. In certain embodiments, the term refers to enhancing a parameter to achieve a desired goal e.g., increasing fertility of an individual or viability of an embryo.

The term “ameliorate” as used herein, unless otherwise specified, means to eliminate, delay, or reduce the prevalence of a condition (e.g., zygote arrest or infertility) or severity of symptoms associated with a condition or disease.

The term “an effective amount” and a “therapeutically effective amount” are intended to qualify the amount of an active ingredient (e.g., a CHK1 inhibitor) which will achieve the goal of preventing or treating a disease or condition or that which will achieve the goal of decreasing the risk that the patient will suffer an adverse health event (e.g., unwanted infertility, zygote arrest), while avoiding adverse side effects such as those typically associated with alternative therapies. The term also refers to the amount of an additive in a culture medium that can promote/enhance embryo development.

The terms “treating” and “treatment” as used herein, unless otherwise specified, includes delaying the onset of a condition, reducing the severity of symptoms of a condition, or eliminating some or all of the symptoms of a condition.

The general inventive concepts are based, in part, on the recognition that specific enhanced kinase activity can block embryonic development and that the expression of CHK1 plays a role in human (in)fertility. This is based on the discovery that that CHK1 mutations show increased kinase activity and the application of a CHK1 inhibitor was able to significantly rescue the phenotype in both mouse and human. While not wishing to be bound by theory, Applicants have demonstrated that dominant mutations in CHK1 are responsible for pronuclear fusion failure and zygote arrest (PFF-ZA).

Further, Applicants have demonstrated that exposure to a CHK1 inhibitor (i.e., in a culture medium) can substantially enhance blastocyst development while not interfering with embryo quality. This is important in as much as it is known that blastocyst development can be increased, but often this comes at the expense of decreasing embryonic quality, an unwanted outcome in the field of infertility.

More particularly, Applicants demonstrated that mutations in CHK1 were responsible for PFF-ZA in 7 out of 29 cases, likely through increasing the CHK1 activity. Importantly, PFF-ZA caused by these mutations was reduced or treated by exposure to a CHK1 inhibitor. Applicant have also demonstrated that exposure to a medium containing a certain concentration of an additive (e.g., a CHK1 inhibitor) can lead to enhanced (improved/increased) blastocyst development without loss of embryo quality. In certain exemplary embodiments, the CHK1 inhibitor is selected from Rabusertib, CCT245737, Prexasertib, AZD7762, and PF477736. In certain exemplary embodiments, the CHK1 inhibitor is PF477736. The structure of an exemplary CHK1 inhibitor is shown below:

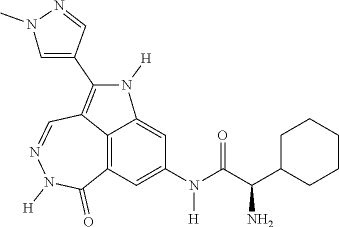 [SMILES: Cn1cc(-c2[nH]c3cc(NC(=O)[C@H](N)C4CCCCC4)cc4c3c2C=NNC4=O)cn1]

Accordingly, the general inventive concepts recognize a method for the treatment of infertility comprising, identifying an individual having altered CHK1 function, and administering therapeutically effective amount of a CHK1 inhibitor.

The general inventive concepts also relate to a culture medium for mammalian embryo culturing, the culture medium comprising a therapeutically effective amount of a CHK1 inhibitor. In certain embodiments, the additive is a CHK1 inhibitor in an amount of 0.1 nM to 100 nM.

The general inventive concepts also relate to and contemplate a method for enhancing embryonic development. The method comprises providing a culture medium, adding a therapeutically effective amount of a CHK1 inhibitor and contacting the embryo with the culture medium. In certain exemplary embodiments, embryonic development is enhanced by modulating blastocyst development rate.

The general inventive concepts also relate to and contemplate a method for the treatment and/or prevention of zygote arrest, the method comprises identifying a subject suffering from zygote arrest or at increased risk of zygote arrest, contacting a zygote from the individual with a therapeutically effective amount of a CHK1 inhibitor. In certain exemplary embodiments, the therapeutically effective amount corresponds to an amount sufficient to overcome the zygote arrest.

The general inventive concepts also relate to and contemplate a composition for the treatment and/or prevention of zygote arrest, the composition comprises a therapeutically effective amount of a CHK1 inhibitor.

The general inventive concepts also relate to and contemplate method for treating altered kinase activity, the method comprising identifying an individual having altered CHK1 function, and administering therapeutically effective amount of a CHK1 inhibitor. In certain embodiments the individual has increased kinase activity resulting in zygote arrest and/or infertility.

In certain exemplary embodiments, an individual is identified as having altered CHK1 function by measuring an expression level of CHK1 and determining whether the level surpasses a threshold value determined from a healthy or otherwise fertile population. In an exemplary embodiment, an individual is identified as having altered CHK1 function by genetic identification of a mutation. In certain embodiments, the mutation results in an increased expression level of CHK1, including an increase to a level above a threshold value.

Any one of the culture additives (i.e., CHK1 inhibitors) described in the present disclosure can be used in the methods or compositions described herein, including conventional embryo culture, methods for enhancing embryonic development, method for the treatment of infertility, method for the treatment and/or prevention of zygote arrest, and treating altered CHK1 function, among others.

As the general inventive concepts demonstrate, in certain embodiments (e.g., culture medium) the concentration of any additive, such as a CHK1 inhibitor, should be regulated to achieve the desired result (e.g., enhanced embryo development rate), while avoiding issues with embryo quality. In certain exemplary embodiments, the concentration of the additive (e.g., a CHK1 inhibitor) is 0.1 nM-100 nM. In certain exemplary embodiments, the concentration of the additive is 0.1-10 nM. In certain exemplary embodiments, the concentration of the additive is 0.1 nM-20 nM. In certain exemplary embodiments, the concentration of the additive is 0.1 nM-30 nM. In certain exemplary embodiments, the concentration of the additive is 0.1 nM 40 nM. In certain exemplary embodiments, the concentration of the additive is 0.1 nM-50 nM. In certain exemplary embodiments, the concentration of the additive is 0.1 nM-60 nM. In certain exemplary embodiments, the concentration of the additive is 0.1 nM-70 nM. In certain exemplary embodiments, the concentration of the additive is 0.1 nM-80 nM. In certain exemplary embodiments, the concentration of the additive is 0.1 nM-90 nM. In certain exemplary embodiments, the concentration of the additive is 0.2 nM-100 nM. In certain exemplary embodiments, the concentration of the additive is 0.3 nM-100 nM. In certain exemplary embodiments, the concentration of the additive is 0.4 nM-100 nM. In certain exemplary embodiments, the concentration of the additive is 0.5 nM-100 nM. In certain exemplary embodiments, the concentration of the additive is 0.6 nM-100 nM. In certain exemplary embodiments, the concentration of the additive is 0.7 nM-100 nM. In certain exemplary embodiments, the concentration of the additive is 0.8 nM-100 nM. In certain exemplary embodiments, the concentration of the additive is 0.9 nM-100 nM.

Through the use of whole-exome sequencing (WES) Applicants have screened the candidate gene contributing to human zygote arrest and identified CHK1 (MIM: 603078; GenBank: NM_001274.5) mutations in four independent families.

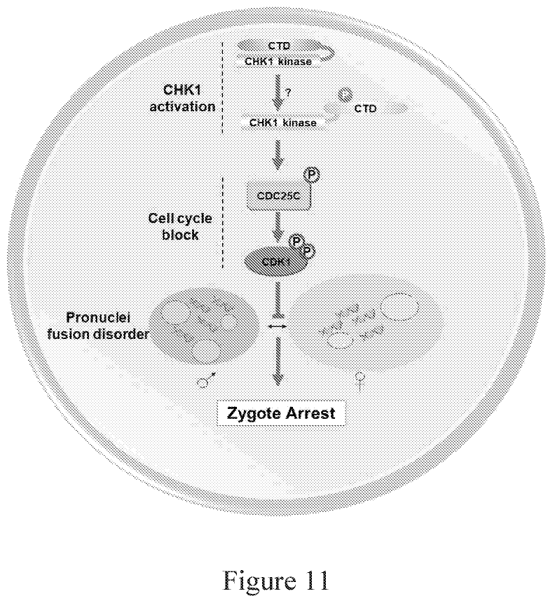

A recent study has shown that human zygotic cleavage failure is a Mendelian genetic disorder, but the etiology of most patients remains unknown. The general inventive concepts are based, in part, on the discovery that CHK1 mutations could cause human zygote arrest that is mainly characterized by a pronuclei fusion disorder in a pattern of female-restricted autosomal dominant inheritance. CHK1 is highly expressed in the zygote stage compared to other pre-implanted stages both in mouse and human. These activated mutations are in the C-terminal domain of the CHK1 protein and have a specific effect on the zygotes. Specifically, these mutations changed the protein structure, altered the protein localization and caused cell cycle arrest through the inhibitory phosphorylation of CDC25C/CDK1 pathway (FIG. 11).

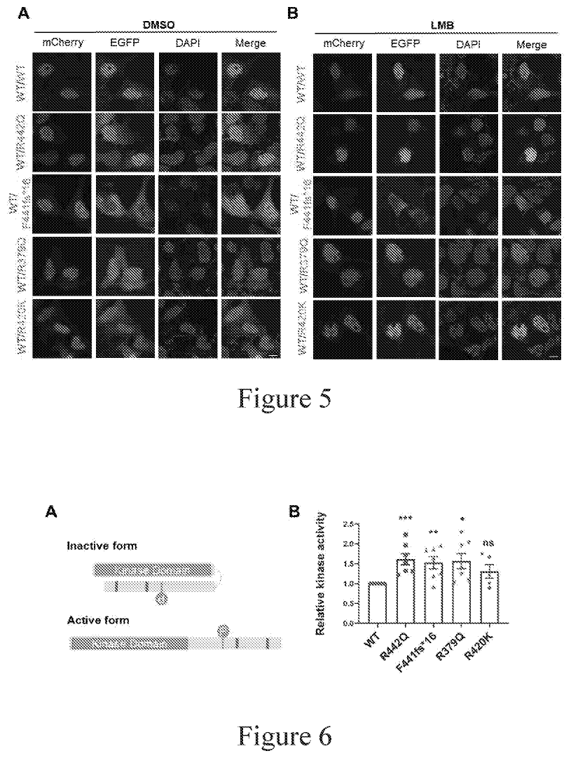

The kinase domain of human CHK1, which lacks the C-terminal domain including the ATR phosphorylation site (S345), showed stronger catalytic activity than the full-length CHK1 protein in vitro. Furthermore, it is generally accepted that CHK1 drives the transition between activation and inactivation in an auto-inhibitory mode. In the absence of DNA damage, the N-terminal of CHK1 interacts with its C-terminal to maintain an inactivated state that forms a closed structure; when DNA damage signals appear, the upstream kinase (ATR) phosphorylates CHK1 at S345 to trigger its open structure, which is followed by the activation of CHK1 (FIG. 6A). These mutations are likely to destroy the closed conformation of CHK1 in order to release its intramolecular self-inhibition, exposing the kinase domain and leading to CHK1 activation.

CHK1 inhibitors have been widely reported to increase the sensitivity to tumor treatments in combination with other anticancer agents. Since CHK1 mutants had increased kinase activity, we chose a selective ATP-competitive inhibitor, PF477736, to inhibit the increased activity. This made it possible to overcome zygote arrest in mouse through down-regulation of phosphorylated CDC25C and CDK1. In order to further explore the efficiency of PF477736, applicants explored an optimal concentration in mouse eggs to harvest the blastocyst, and the subsequent application of PF477736 significantly improved the blastocyst yields of zygotes carrying CHK1 mutations and produced heathy mouse offspring. Furthermore, the frozen zygotes of one patient (IIM-2 in Family 1), which had been cultured to Day 3 without evidence of division, underwent cleavage after treatment with PF477736.

Applicants have identified a new genetic cause of female infertility and shed light on the key role played by CHK1 in the transition from accomplishing oocyte meiosis to initiating embryo mitosis, beginning a new life. The application of a CHK1 inhibitor in the blocked zygotes of patients, and the confirmation of its efficiency in mouse fertilized eggs, offers a potential intervention for the treatment of this kind of disease, which will be the first step towards precise treatment of infertile patients suffering from zygote arrest.

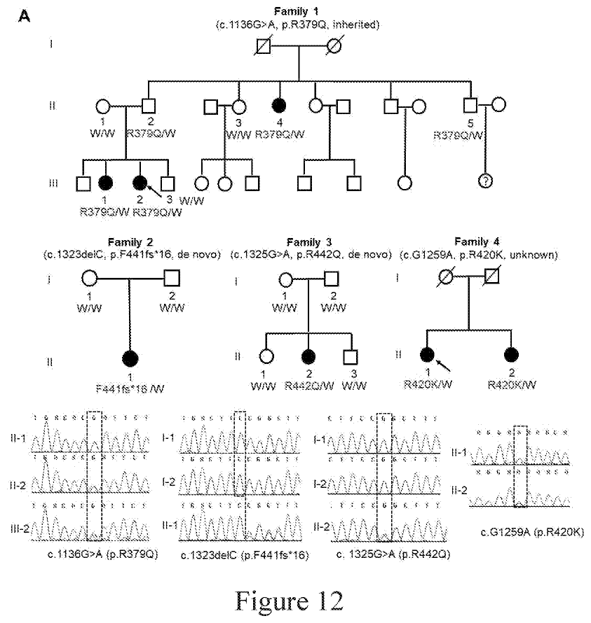

Applicants identified a three-generation family (Family 1) with female primary infertility (FIG. 12A). The proband underwent three IVF or ICSI cycles and obtained 24 zygotes. The majority of these fertilized eggs held distinct pronuclei on the first cleavage day (Day 1), and the female and male pronuclei did not fuse until the third cleavage day (Day 3); whereas the control individuals had already divided on Day 1, and proceeded to the eight-cell stage on Day 3 (Table 1). Importantly, the elder sister and an aunt of the proband also suffered from infertility (FIG. 12A). WES analysis in Family 1 (III-2, II-1 and II-3) uncovered a heterozygous missense mutation c.1136G>A (p.R379Q) in CHK1, which co-segregated with female infertility and presented an autosomal dominant inheritance pattern with paternal transmission, though the father was not affected (FIG. 12A).

TABLE 1

| | | | | | | | | Fertilized | Embryos |
|---|---|---|---|---|---|---|---|---|---|
| | | | Duration of | IVF/ | | | | oocytes | that |
| | | Age | infertility | ICSI | Retrieved | Mature | Fertilized | states | could be |
| Patient | Mutation | (Years) | (Years) | cycles | oocytes | oocyte | oocytes | on Day 1 | transferred |
| III-2 in | R379Q | 28 | 3.5 | 1-IVF | 11 | 10 | 10 | 8 in PN, 2 in 1C | 0 |
| Family 1 | | | | 2-ICSI | 15 | 13 | 9 | 1 in PN, 8 in 1C | 0 |
| | | | | 3-ICSI | 8 | 5 | 5 | 5 in PN | 0 |
| II-1 in | F441fs*16 | 31 | 7 | 1-IVF | 13 | 12 | 11 | 11 in PN | 0 |
| Family 2 | | | | 2-ICSI | 12 | 9 | 8 | 6 in PN, 2 in 1C | 0 |
| | | | | 3-ICSI | 7 | 7 | 6 | 6 in PN | 0 |
| II-2 in | R442Q | 27 | 5 | 1-IVF | 17 | 17 | 15 | 15 in PN | 0 |
| Family 3 | | | | 2-ICSI | 8 | 7 | 4 | 2 in PN, 2 in 1C | 0 |
| | | | | 3-ICSI | 5 | 5 | 5 | 5 in PN | 0 |
| | | | | 4-ICSI | 7 | 6 | 2 | 2 in PN | 0 |
| II-1 in | R420K | 36 | 7 | 1-IVF | 9 | 7 | 5 | 2 in PN, 3 in 1C | 0 |
| Family 4 | | | | 2-ICSI | 7 | 7 | 5 | 3 in PN, 2 in 1C | 0 |
| II-2 in | R420K | 32 | 10 | 1-IVF | 5 | 3 | 3 | 3 in PN | 0 |
| Family 4 | | | | 2-ICSI | 8 | 8 | 8 | 8 in 1C | 0 |

*IVF denotes in vitro fertilization, ICSI intracytoplasmic sperm injection, Day 1 the first cleavage day, PN pronucleus, C cell

Applicants subsequently found three other CHK1 mutations using Sanger sequencing or WES in four infertile women among 26 patients exhibiting a similar zygote arrest phenotype: one patient in Family 2 (c.1323delC, p. F441fs*16, de novo), one in Family 3 (c.1325 G>A, p.R442Q, de novo) and two in Family 4 (c.1259 G>A, p. R420K, unknown) (FIG. 12A). Haplotype analysis proved the paternity relationship between the patients and their parents in Family 2 and Family 3, and also indicated that the disease allele origins of the four families were totally independent. The four patients in Families 2, 3 and 4 underwent at least two failed IVF or ICSI attempts, and most of their zygotes arrested with obvious female and male pronuclei (Table 1).

The mutations identified were neither found in database of Genome Aggregation Database (gnomAD) and 1000 Genomes Browser (1000g_All), nor in 300 healthy female controls. With the exception of the truncated mutation F441fs*16, the remaining three mutations (R379Q, R442Q and R420K) were all predicted to affect the function of the CHK1 protein by SIFT, Polyphen-2 and Mutation Taster. Moreover, all the identified mutations were classified as likely pathogenic according to the criteria of the ACMG (The American College of Medical Genetics and Genomics), see Table 2.

TABLE 2

| | | cDNA | Protein | Mutation | | | Mutation | | | |
|---|---|---|---|---|---|---|---|---|---|---|
| Family | Pattern | change | change | type | SIFT^{a} | PPH2^{a} | Taster^{a} | 1000g_A ^{b} | gnomAD^{b} | ACMG^{c} |
| 1 | Inherited | c.G1136A | p.R379Q | missense | D | D | D | NA | NA | LP |
| 2 | de novo | c.1323delC | p.F441fs*16 | frameshift | NA | NA | NA | NA | NA | LP |
| | | | | deletion | | | | | | |
| 3 | de novo | c.G1325A | p.R442Q | missense | D | D | D | NA | NA | LP |
| 4 | unknown | c.G1259A | p.R420K | missense | D | P | D | NA | NA | LP |

Abbreviations are as fllows: D, damaging; P, probably damaging; NA, notavailable; LP: Likely pathogenic.
^{a}Mutation assessment by SIFT, Polyphen-2 (PPH2) and Mutation Tester.
^{b}Allele frequency of corresponding mutations in all population of 1000 Genomes (1000g_E) and gnomAD database.
^{c}Mutation assessment according to the criteria of the American College of Medical Genetics and Genomics.

### Expression of Human CHK1 Mutations in Mouse Zygotes

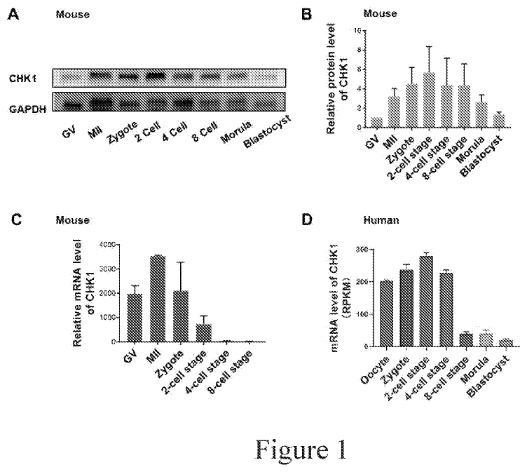

Applicants western blotting and real-time quantitative PCR results of CHK1 in mouse oocytes and pre-implanted embryos demonstrated that the expression level of CHK1 was high right before and after fertilization until the 2-cell stage, decreasing after the 4-cell stage (FIG. 1A-C). The published human oocytes and early embryos RNA-Seq data of CHK1 implicated similar results that the mRNA level of CHK1 was relatively high before 8-cell stage (FIG. 1D), suggesting that CHK1 might play an important role in the very early embryo stages.

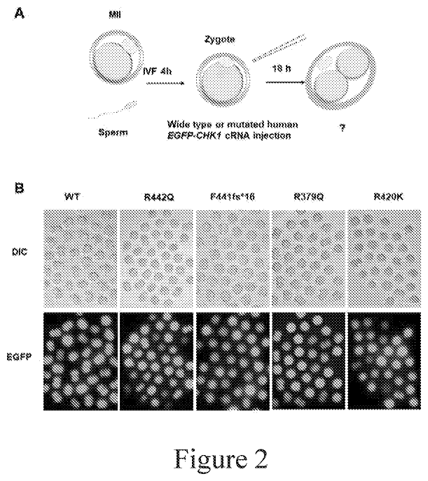

In order to validate the relationship between the identified CHK1 mutations and the zygote arrest phenotype, Applicants injected mutant EGFP-Human-CHK1 complementary RNAs (cRNAs) into mouse fertilized eggs and the cleavage rates were evaluated 18 hours later (FIG. 2A). Applicants results showed that the cleavage rate of mouse zygotes with human CHK1 (hCHK1) mutations decreased significantly compared with wild-type hCHK1, especially in the case of the truncated mutation from Family 2 (8.5% vs. 75.5%) (FIGS. 13A and 2B). At the same time, immunofluorescence results showed that when the zygotes carrying wild-type hCHK1 developed to a 2-cell stage embryo, the male and female pronuclei in the mutant groups had not yet fused (FIG. 13B), in accordance with the phenotype of the patients. These results indicate that the pathogenic mutations of hCHK1 could cause zygote arrest.

### Structural Implications

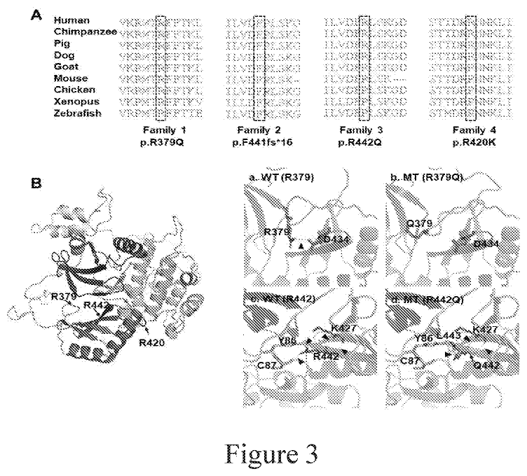

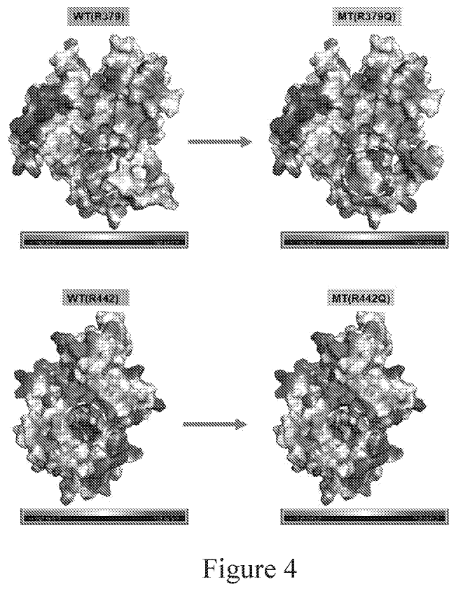

The CHK1 N-terminal is an extremely conserved kinase domain, while the C-terminal is a regulatory domain containing a Ser/Thr (SQ) motif and two highly conserved motifs (CM1 and CM2) (FIG. 6A). The four mutant amino acid residues at R379, F441, R442 and R420 are in or near the two conserved motifs (FIG. 13C) and are highly conserved among different species (FIG. 3A). According to the three-dimensional structure prediction of CHK1 (FIG. 3B), R379 could form a hydrogen bond with a surrounding residue, while the hydrogen bond disappears after replacement of Q379 (FIG. 3B a and b). R442 can form four hydrogen bonds with the surrounding residues while the hydrogen bonds between R442 and the two residues (Y86 and C87) in the N-terminal domain disappear after being replaced by Q442, accompanied by forming a new hydrogen bond with L443 (FIG. 3B c and d). The substitution of residue R by Q also caused a change in the surface potential of the protein (FIG. 4), probably owing to the fact that R is a basic amino acid while Q is a neutral amino acid. While not wishing to be bound by theory, Applicants inferred that the structural changes might further affect the function of the protein.

### Nuclear-Cytoplasmic Dis-Location of CHK1 Mutants

CHK1 locates on the chromatin of nuclei under normal condition; when activated, CHK1 dissociates from the chromatin, binds to 14-3-3 protein in the nucleoplasm and a proportion is exported to the cytoplasm in order to regulate both the nuclear and cytoplasmic checkpoints. CM1 and CM2 respectively correspond to the nuclear export signal (NES) and the nuclear localization signal (NLS) of CHK1 and mutations in or near these conserved regions can affect subcellular localization of the protein and even its checkpoint function. It should be emphasized that the mutation p.R379Q in Family 1 is located in the NES region, while the mutations p.F441fs*16 (Family 2), p.R442Q (Family 3) and p.R420K (Family 4) are all located in the NLS region (FIG. 13C). In order to explore the effects of these mutations on the intracellular localization of the CHK1 protein, Applicants co-transfected mCherry tagged wild-type CHK1 constructs with EGFP-tagged CHK1 (wild-type or mutant) constructs into HEK-293 cells in a heterozygous pattern. Compared to the wild-type control (WT), all of the mutants increased cytoplasmic signals (FIGS. 13D and 13E), especially the truncated mutation p. F441fs*16 with almost complete loss of expression in the nucleus (FIG. 13D).

Protein nuclear export is usually regulated by Crm1 which binds to the NES of substrate, and nuclear export of CHK1 is Crm1-dependent. After treatment with Leptomycin B, a Crm1 inhibitor, the cytoplasmic localization of the mutation p.R379Q, p.R442Q and p.R420K disappeared (FIG. 5), indicating that the cytoplasmic localization of the three mutations were mainly driven by NES rather than NLS. However, the mutant p.F441fs*16 still showed cytoplasmic localization after the treatment with LMB (FIG. 5), indicating that the cytoplasmic localization of the truncated protein was indeed the result of NLS disruption.

All in all, the four pathogenic mutations located in the C-terminal regulatory domain of CHK1 changed the nuclear and cytoplasmic localization of the protein, which was related to the nuclear export signal region and nuclear localization signal region where the mutations located. While not wishing to be bound by theory, Applicants believe the location of CHK1 is closely related to its intracellular function.

### Effects of CHK1 Mutations on CDC25C/CDK1 Pathway

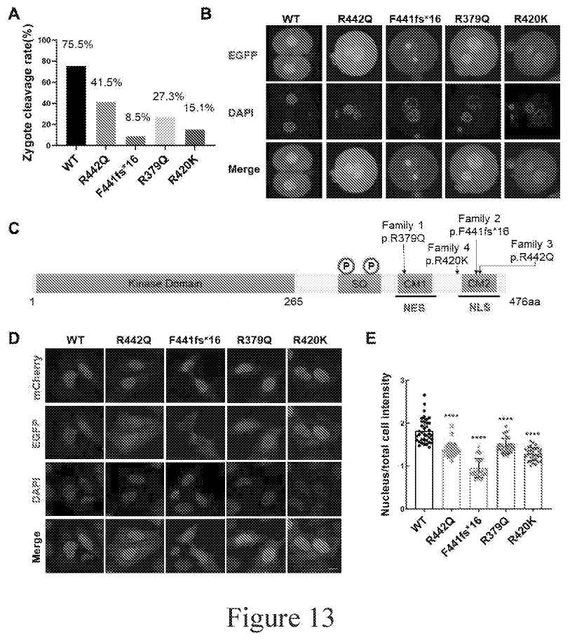

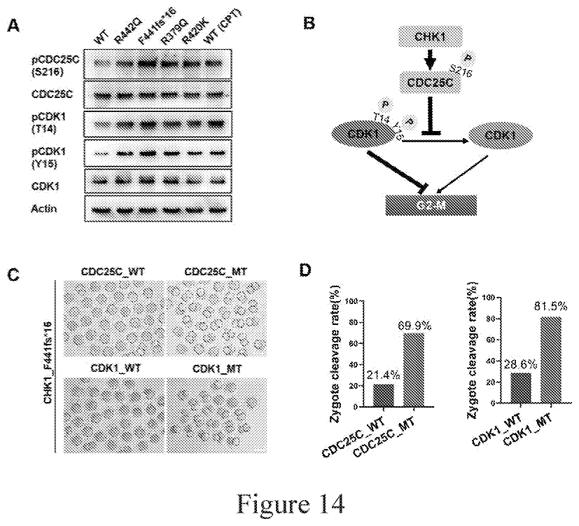

Activated CHK1 can directly phosphorylate CDC25C at S216, resulting in reduced degradation of inhibitory phosphorylation of CDK1 at both T14 and Y15, thus preventing the G2/M transition and causing cell cycle arrest (FIG. 14B). Moreover, the late pronucleus stage of the zygote corresponds to the G2 stage, after which the zygote enters the mitosis stage. To further explore the effects of CHK1 mutations on the cell cycle, Applicants evaluated the kinase activity of WT and mutant CHK1, and showed that the mutants held increased kinase activity compared with the WT (FIG. 6B). After this, Applicants detected the expression of downstream CHK1 effectors in HEK-293T cells via Western Blot analysis. As expected, CHK1 mutants increased the expression of phosphorated CDC25C (S216) and CDK1 (T14 and Y15) similar with the result under the treatment with CPT (a DNA damage drug to active CHK1) and the truncated mutation had the strongest effect on the accumulation of inhibitory pCDKIs (FIG. 14A). It has been previously reported that inhibition of CDK1 seriously inhibited the migration and fusion of male and female pronuclei in starfish fertilized eggs. Hence, we infer that CHK1 mutants might interfere with pronuclei fusion of fertilized eggs by producing more inhibitory pCDK1.

To further assess the effects of CHK1/CDC25C/CDK1 pathway on zygote arrest, Applicants overexpressed the mutation p.F441fs*16 in mouse zygotes, along with mutated CDC25C or CDK1 lacking their phosphorylation sites. Applicants discovered that both mutated CDC25C (CDC25C_MT) and CDK1 (CDC25C_MT) were able to overcome p.F441fs*16-induced zygote cleavage failure (FIGS. 14C and 14D). Overall, these results confirmed that CHK1 mutations had higher activity and could cause zygote arrest through phosphorylation and inhibition of the CDC25C/CDK1 pathway.

### Rescue Through a CHK1 Inhibitor

The C-terminal CHK1 mutants presented an activated function and hold higher kinase activity, which lead to cell cycle arrest through the phosphorylation of downstream factors. Therefore, it is possible to rescue zygote block resulting from increased activity of CHK1 by applying one of its inhibitors. PF477736, a selective ATP-competitive CHK1 inhibitor, has been previously employed to inhibit CHK1's activity in a clinical trial to treat the tumor combined with gemcitabine, an anti-tumor drug.

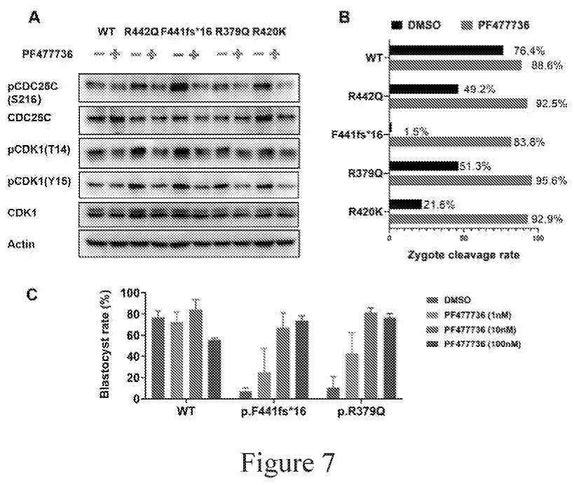

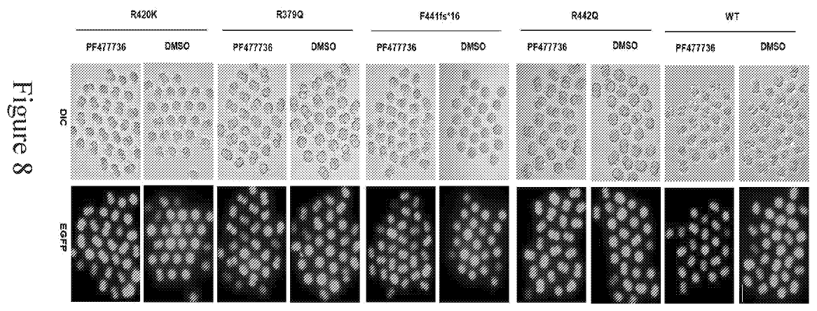

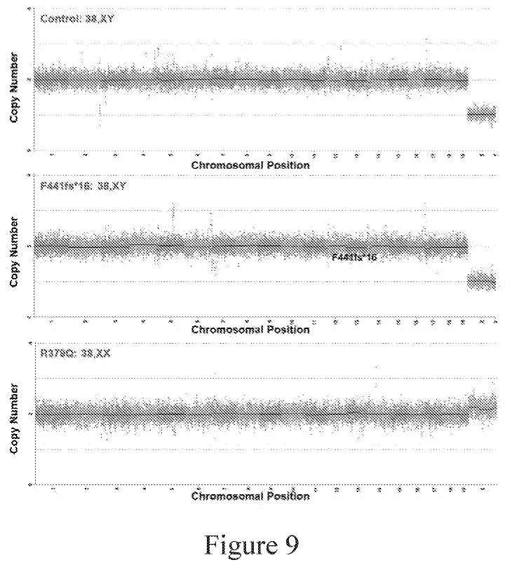

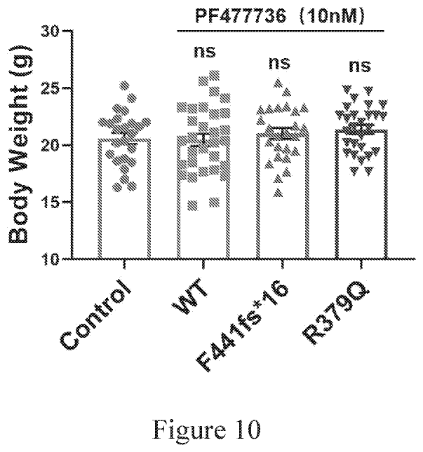

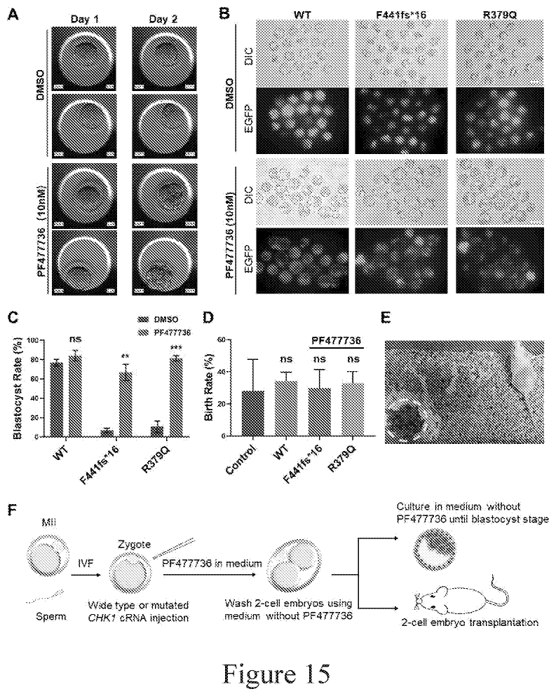

In this study, Applicants observed that PF477736 could decrease the expression levels of pCDC25C and pCDK1s, two downstream CHK1 proteins, in HEK-293T cells (FIG. 7A). It was also able to dramatically increase the cleavage rate of zygotes with mutations in contrast to DMSO (92.5% vs. 49.2% in mutant R442Q; 83.8% vs. 1.5% in mutant F441fs*16; 95.6% vs. 51.3% in mutant R379Q; 92.9% vs. 21.6% in mutant R442Q) (FIGS. 7B and 8), using a 10 nM concentration. Applicants further explored the effective concentration of PF477736 and found that the concentration of 10 nM had the best efficiency to increase the blastocyst yields (66.7% vs. 6.8% in mutant F441fs*16; 81.3% vs. 10.7% in mutant R379Q) of the concentrations tested, when compared to DMSO in mutated groups (FIGS. 7C and 15B-C). In addition, Applicants randomly selected nine PF477736-treated blastocysts carrying mutated hCHK1 (F441*fs or R379Q) and detected no aneuploidy (FIG. 9). The treated embryos were then transferred to pseudo-pregnant female mice (FIG. 15F), which were able to generate healthy offspring (FIG. 4E). There was no significant difference in birth rate in the PF477736 (10 nM) treated mutant groups compared to completely normal controls (Table 3 and FIG. 15D), and the litters showed normal growth and weight (FIG. 10).

TABLE 3

| | Zygote | Transfer | Recipient | | Birth |
|---|---|---|---|---|---|
| Group | number | number | number | Pups | rate |
| Control | 100 | 50 | 2 | 7 | 14% |
| | | 50 | 2 | 21 | 42% |
| WT | 100 | 50 | 2 | 19 | 38% |
| | | 50 | 2 | 15 | 30% |
| F441fs*16 | 97 | 47 | 2 | 10 | 21% |
| | | 50 | 2 | 19 | 38% |
| R379Q | 97 | 50 | 2 | 19 | 38% |
| | | 47 | 2 | 13 | 28% |

Human zygote testing: Donated frozen zygotes from patient III-2 (Family 1), which had extended culture until Day 3 after fertilization without division, were thawed and treated with 10 nM PF477736. Surprisingly, Applicants found that these blocked zygotes were able to divide and develop when treated with PF477736 (FIG. 15A). Therefore, PF477736 can contribute to the rescue of zygote cleavage failure by inhibiting the activity of mutant CHK1, which provides a potential novel clinical intervention for patients carrying CHK1 mutations.

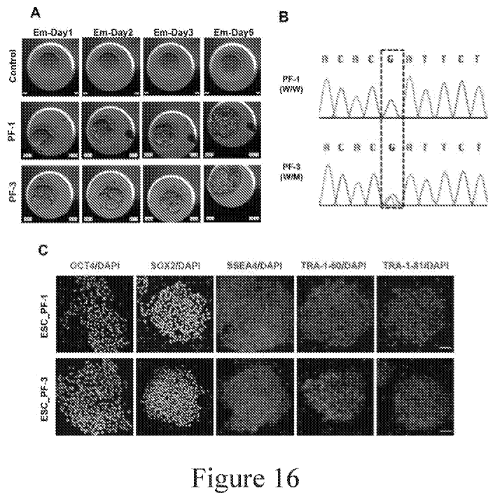

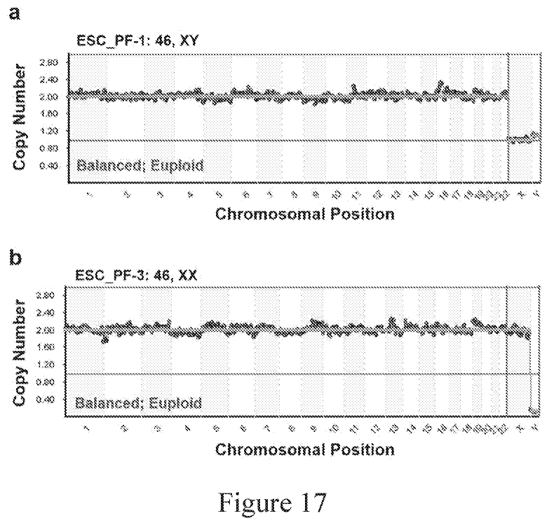

Five fresh fertilized eggs donated by the same patient were also treated with PF477736 right after the formation of pronuclei. Applicants observed that whereas the two untreated control zygotes remained in the pronuclei stage and never divided as expected, all five zygotes treated with PF477736 overcame one cell stage and two of them even developed into good-quality blastocysts (FIG. 16 A, PF-1 and PF-3, Table 4). Genotyping analysis showed one blastocyst was WT while the other carried R379Q mutation (FIG. 16B). Furthermore, both of the two blastocysts were successfully derived into human embryonic stem cells (FIG. 16C, ESC_PF-1 and ESC_PF-3) exhibiting pluripotency and genetic testing proved their genetic integrity (FIG. 17).

TABLE 4

| | Control | | Treated with PF477736 | | | | |
|---|---|---|---|---|---|---|---|
| Embryo ID | Ctrl-1 | Ctrl-2 | PF-1 | PF-2 | PF-3 | PF~4 | PF-5 |
| Zygote | 2PN | 2PN | 2PN | IPN | 2PN | 2PN | 2PN |
| Em-D1 | UD | UD | 2C3′ | 2C3′ | 2C3′ | 1C | 2C2′ |
| Em-D2 | UD | UD | 4C4′ | 2C3′ | 4C4′ | 4C3′ | 2C2′ |
| Em-D3 | UD | UD | 8C3′ | 8C2′ | 8C3′ | 6C2′ | 4C2′ |
| Em-D5/6 | UD | UD | 4BB | D | 4AA | D | D |

“Em” represents embryo;
“D” represents day;
“UD” represents undivided;
“D” represents degenerated

Derivation of Human Embryonic Cell (hESC) Lines

The inner cell mass (ICM) of the patient's blastocysts (PF-1 and PF-3) produced by treatment with PF477736 were planted on mitotically inactivated mouse embryonic fibroblasts (MEF) in modified human embryonic stem cell culture medium¹ in a humidified incubator at 37° C., 6% CO₂ 5% O₂. Culture medium was usually changed every day. Outgrowths were formed after five days and passaged on fresh MEF feeders followed by mechanically separating into several pieces.

### Human Subjects

Patients with familial or sporadic zygote arrest, as well as healthy control individuals were recruited in the Center for Reproductive Medicine, Shandong University. All subjects signed informed consents, and this study was reviewed and approved by the Institutional Review Board of Reproductive Medicine, Shandong University.

The proband (III-2) in Family 1 was 28 years old and had been infertile for 3.5 years without contraception. She had regular menstrual cycles with normal sex hormone level and the sperm count, morphology and motility of her spouse were normal too. She was diagnosed as primary infertility. Three IVF/ICSI cycles were performed and a total of 24 fertilized eggs were obtained. However, the majority of the zygotes arrested in the pronuclei (PN) or 1-cell stages on the first day after fertilization when the embryos from normal controls are usually in 2-cell stage. Almost none of them divided in the next three days, resulting in no transferable embryos. We regard this phenotype as zygote arrest chiefly characterized by pronuclei fusion failure (PFF-ZA). What's more, it is worth noting that an elder sister and an aunt of the patient also suffered from infertility.

The patient (II-1) in Family 2 was 31 years old. Although the menstrual cycle and sex hormone levels were normal, she had a 7-year history of primary infertility. Then she tried three IVF/ICSI cycles in our center, and a total of 25 fertilized eggs were obtained. On the first day of cleavage, 23 fertilized eggs still had PN and only 2 eggs were in 1-cell stage. Almost all of them did not divide and still showed clear PN in the next few days, which was much more serious than the condition of patient in Family 1.

The third patient (11-2) we found in Family 3 was 27 years old. Similar to the former two patients, she had a 5-year history of primary infertility with regular menstrual cycle and normal sex hormones. She had four IVF/ICSI cycles and obtained a total of 26 fertilized eggs, of which 23 fertilized eggs were blocked at PN stage and only 2 eggs in 1-cell stage on the first day of cleavage. No transferable embryo could be used either.

The proband (II-1) in Family 4, 36 years old, had a 7-year history of infertility with normal menstrual cycle and sex hormone levels, diagnosed as primary infertility. She performed two IVF/ICSI cycles and a total of 10 fertilized eggs were obtained, among which five were in PN in the first cleavage day and the others were in 1-cell stage. Most of the embryos were not divided and there were no transferable embryos as well. The younger sister of the patient with a 10-year history of infertility had two failed IVF/ICSI treatments and also showed zygote arrest.

### Whole Exome Sequencing, Data Analysis and Validation

The DNA of human peripheral blood was extracted by QIAamp DNA Mini Kit according to the manufacturer's instruction. Exome capture and sequencing were performed using Agilent SureSelect Whole Exome capture and Illumina platform. A variant was considered to be a candidate mutation if it 1) had not been reported previously or had a prevalence below 0.01% in the three public databases (dbSNP, 1000 Genome, and gnomAD); 2) was a non-synonymous SNP/insertion/deletion in the coding region or in splicing region; 3) was predicted to be harmful via at least two software, such as SIFT, Polyphen-2 and Mutation Taster. Next, the filtered candidate mutations were verified by sanger sequencing in the family members, excluding ones that were not co-segregated with the disease. Finally, the candidate gene were further verified in 300 fertile women in our center to remove the loci in normal control. See the primers in the following Table.

TABLE 5

| Primers used for site- | |
|---|---|
| directed mutagenesis | Sequence |
| CHK1-LF097-F | GGTTGGTCAAAAGAATGACACAATTCTTTACCAAATTGGATGC |
| CHK1-LF097-R | GCATCCAATTTGGTAAAGAATTGTGTCATTCTTTTGACCAACC |
| CHK1-LF057-F | AGAAATGGATGATAAAATATTGGTTGACTTCGGCTTTCTAAGGTATTTT |
| CHK1-LF057-R | AAAATACCTTAGAAAGCCGAAGTCAACCAATATTTTATCATCCATTTCT |
| CHK1-LF033-F | GGATGATAAAATATTGGTTGACTTCCAGCTTTCTAAGGTATTTTTATGTTTTA |
| CHK1-LF033-R | TAAAACATAAAAATACCTTAGAAAGCTGGAAGTCAACCAATATTTTATCATCC |
| CHK1-LF160-F | GGTTACTATATCAACAACTGATAGGAAAAACAATAAACTCATTTTCAAAGTGA |
| CHK1-LF160-R | TCACTTTGAAAATGAGTTTATTGTTTTTCCTATCAGTTGTTGATATAGTAACC |
| CDC25C-F | AGTTCTCTGGCATCGCCGGGGAGCGATATAG |
| CDC25C-R | CTATATCGCTCCCCGGCGATGCCAGAGAACT |
| CDK1-F | CCCTTATACACAACTCCAGCGGCACCTTCTCCAATTTTCTCTATTTTGGTATAATCTT |
| CDK1-R | AAGATTATACCAAAATAGAGAAAATTGGAGAAGGTGCCGCTGGAGTTGTGTATAAGGG |
| Primers used for qRT- | |
| PCR | Sequence |
| CHK1-RT-F | GATCATCAGCAATGCCTCCT |
| CHK1-RT-R | TTCAGCTCAGGGATGACCTT |
| GAPDH-RT-F | CTGCACCACCAACTGCTTAG |
| GAPDH-RT-R | GGATGCAGGGATGATGTTCT |

Applicants identified seven patients in four independent families carrying heterozygous CHK1 mutations (Family 1: c.1136G>A, p.R379Q, inherited; Family 2: c.1323delC, p.F441fs*16, de novo; Family 3: c.1325 G>A, p.R442Q, de novo; Family 4: c.1259 G>A, p. R420K, unknown). Haplotype analysis proved the paternity relationship between the patient and their parents in Family 2 and Family 3. In addition, no matter zygotes carrying the CHK1 mutations or not, they were all arrested at zygote stage and never divided, indicating maternal factor may contribute to the phenotype.

### Expression Construction

Primers were designed to amplify the target gene from pENTER vector (Vigene Biosciences) containing the full-length coding sequence of human CHK1 (NM_001274). Then the CHK1 gene was cloned into the pcDNA3.1 (+) vector together with the enhanced green fluorescent protein (EGFP) or red fluorescent protein (mCherry) coding sequence, in order to obtain the CHK1 fusion protein with green or red fluorescent protein tag at the N-terminal. According to the manufacturer's method, the plasmid containing the coding sequence of EGFP and CHK1 was mutagenized by Quick Change Lightning Site-Directed Mutagenesis Kit (Agilent Technologies) to obtain CHK1 mutated plasmids (c.G1136A, c.1323delC c.G1325A, and c.G1259A). The mutant plasmids CDC25C (BC019089.2) and CDK1 (NM_001786.4) were obtained with the same kit. The primers for site-directed mutation can be found in Table 4.

### Mouse Oocytes/Embryos Collection

The 6-8 weeks healthy ICR female mice (Beijing Vital River Laboratory Animal Technology Co.) were super-stimulated with 7.5 IU pregnant mare's serum gonadotropin (PMSG, NINGBO SANSHENG) followed by 7.5 IU human chorionic gonadotropin (HCG, NINGBO SANSHENG) after 44-48 h. We then collected cumulus oocyte complex (COC) in the ampulla of mouse oviduct 18 hours later. Sperm from the cauda epididymidis of 8-12 weeks ICR male mice (Beijing Vital River Laboratory Animal Technology Co.) were capacitated in G-IVF medium (Vitrolife) for 1 hour. Then the harvested COC and capacitated sperm were added to new G-IVF medium covered with mineral oil for 4 to 6 hours at 37° C. in a 5% CO₂ atmosphere to obtain fertilized eggs, which would be transferred into KSOM medium (Sigma Aldrich) covered with mineral oil later to obtain 2-cell, 4-cell, 8-cell, morula and blastocyst stage embryos. GV oocytes were obtained from mouse ovaries 44 hours after PMSG injection. MU oocytes need to be digested with hyaluronidase (Sigma-Aldrich) to remove granulosa cells.

### Embryo Immunofluorescence

Mouse embryos were fixed in 4% paraformaldehyde (Solarbio) for 30 minutes and permeated in PBS containing 0.3% TritonX-100 for 20 min. After being blocked in 1% bovine serum albumin (BSA, Sigma) in PBS for 1 h, they would be re-stained with 4-methyl-6-methyl-2-phenylindole (DAPI, Vector Laboratories) for 10 minutes. After mounting, oocytes/embryos were examined with a confocal laser-scanning microscope (Zeiss LSM 780, Carl Zeiss AG, Germany).

In Vitro cRNAs Synthesis and Microinjection

The plasmids were linearized with appropriate restriction endonuclease. According to the factory's method, 5′ capped cRNAs were synthesized via mMESSAGE mMACHINE T7 Transcription Kit (Invitrogen, AM1344) and then added with poly (A) tail using Poly(A) Tailing Kit (Invitgen, AM1350), followed by purification with RNeasy MinElute Cleanup Kit (QIAGEN,74204) and dilution in nuclease-free water. About 5 pl cRNA solution (1400 ng/ul) was microinjected into the cytoplasm of the fertilized eggs.

### Molecular Modeling and Evolutionary Conservation Analysis

The three-dimensional structures of CHK1 (NP 001265.2) were predicted by SWISS-MODLE webserver (PDB ID:6C9D). Molecular graphics and analysis were carried out by PyMol software. Evolutionary conservative analysis was performed with Clustalx software.

### Cell Transfection and Immunofluorescence

HEK-293(T) cells were cultured in DMEM/high glucose medium (HyClone, SH30243.01B) with 10% fetal bovine serum (FBS, BI, 04-001-1ACS) at 37° C. with 5% CO₂. When the cell density reached 70% Mo-80% fusion, they would be transfected by Lipofectamine 3000 Transfection Kit (Invitrogen, L3000015) according to the scheme given by the manufacturer.

HEK-293(T) cells were cultured in DMEM/high glucose medium (HyClone, SH30243.01B) with 10% fetal bovine serum (FBS, BI, 04-001-1 ACS) at 37° C. with 5% C02. When the cell density reached 70%-80% fusion, they would be transfected by Lipofectamine 3000 Transfection Kit (Invitrogen, L3000015) according to the scheme given by the manufacturer. HEK-293 cells growing on glass slides (NES,801007), co-transfected with mCherry-WT and EGFP-WT or mutated Chk1 for 48 hours, were rinsed with warm PBS followed by being fixed with 4% paraformaldehyde at room temperature for 20 minutes. After being washed 3 times with cold PBS, they would be permeabilized in PBS containing 0.3% Triton X-100 for 20 min, blocked with 5% BSA in PBS for 1 h, and then re-stained by DAPI for 10 minutes. For embryonic stem cells, they would be incubated with first antibodies overnight at 4° C. after blocking, followed by incubation of second antibodies (invitrogen) for 1 hour at room temperature. The antibodies are shown in Table 7.

### CHK1 Kinase Activity Assay

HEK-293T cells transfected with wild-type or mutant CHK1 constructions were collected after 48 hours, followed by the treatment of 500 nM CPT for another 2 hours. The activity of CHK1 kinase in different groups was detected by 96-well Checkpoint Kinase Activity Assay Kit (STA-414, Cell Biolabs) according to the manufacturer's instructions. The relative kinase activity was expressed by the ratio of OD value (450 nm) of all groups to OD value of WT group.

### Quantitative RT-PCR

Mouse oocytes and embryos at different development stages were applied to obtain cDNA with REPLI-g WTA Single Cell Kit (QIAGEN) according to the manufacturer's instructions. Power SYBR Green Master Mix (Takara) was used for qRT-PCR analysis on Roche 480 PCR system. The relative expression level of CHK1 equals 1000·2^{-ΔCt}, of which Δ Ct=Ct (CHK1)−Ct (GAPDH). See the qRT-PCR primers in the following Table.

TABLE 6

| Primers for exon | |
|---|---|
| sequencing | Sequence |
| CHK1-Exon2-F | GCTGTTAATTTTCGTGGGCA |
| CHK1-Exon2-R | TTCAGTTGCCAAAACCCTTG |
| CHK1-Exon3-F | TGAGAACATAGCAGAAACCACT |
| CHK1-Exon3-R | TCCAATTTCACAGTTGCATGAG |
| CHK1-Exon4_5-F | AAGCCCCATATGTGTTAGTGG |
| CHK1-Exon4_5-R | AGACTTGATTTTGCCTTGTATGG |
| CHK1-Exon6-F | TGATGAGGGGCCTTGCTTTA |
| CHK1-Exon6-R | TCTGGCCAAGAGTGAGACC |
| CHK1-Exon7-F | TGAAGTGCCTCTAAAGTTTCCA |
| CHK1-Exon7-R | TGCTCTGAATATACACTCCCCA |
| CHK1-Exon8-F | ACTCCAAGATACAGCAGCAGA |
| CHK1-Exon8-R | GCTATCATGTGTTGTTGACTTGT |
| CHK1-Exon9-F | ACTCCACACTTTGAACATGTCT |
| CHK1-Exon9-R | TCACACACAAGTTCTCATGCT |
| CHK1-Exon10-F | TCAGGTGGTGTGTCAGAGTC |
| CHK1-Exon10-R | GCCTCCCTCCTCTCTTTCTT |
| CHK1-Exon11-F | GGGAGGCCTTCATGCAAAAT |
| CHK1-Exon11-R | CACCCCAGCCTCCCAAAA |
| CHK1-Exon12-F | CCTGGTCTGAAGCGATCCT |
| CHK1-Exon12-R | AGTCTCTTGTATTGTCACCCAGA |
| CHK1-Exon13-F | AGGAACAGTGATGGGCATGA |
| CHK1-Exon13-R | TGGATAAACAGGGAAGTGAACAC |

### Western Blot

100 oocytes/early embryos or collected HEK-293T cells were lysed in protein lysis buffer containing protein phosphatase inhibitor (Beyotime, P1046) for about 30 min, and then denatured for 10 min at 95° C. The proteins were separated by SDS-polyacrylamide gel electrophoresis (SDS-PAGE) and transferred to PVDF membrane (Millipore). The first antibodies were incubated overnight at 4° C., then the HRP-conjugated secondary antibodies were incubated at room temperature for 1 hour. The membranes were eventually developed by Image Lab gel imaging system (Bio-Rad). The antibodies used are shown in the following Table.

TABLE 7

| | | | Appli- |
|---|---|---|---|
| Antibodies | Source | Identifier | cation |
| OCTA Antibody | Santa Cruz | sc-5279 | IF |
| Sox2 (D6D9) Antibody | Cell Signaling | 3579S | IF |
| | Technology | | |
| SSEA4 Antibody | Abcam | ab16287 | IF |
| TRA-1-60 Antibody | Abcam | ab16288 | IF |
| TRA-1-81 Antibody | Santa Cruz | sc-21706 | IF |
| Beta Actin Monoclonal | Protein tech | 66009-1-Ig | WB |
| Antibody | | | |
| GAPDH Monoclonal Antibody | Protein tech | 60004-1-Ig | WB |
| Anti-CDK1 | Abcam | ab32094 | WB |
| Anti-CDK1 (phospho T14) | Abcam | ab58509 | |
| Phospho-cdc25C (Ser216) | Cell Signaling | 9528S | WB |
| Antibody | Technology | | |
| Anti-Cdc25C | Abcam | ab226958 | WB |
| Anti-p-Cdc2 p34 (pY15.44) | Santa Cruz | sc-136014 | WB |

### Embryo Transfer

For embryo transfer experiment, wild-type or mutant CHK1 cRNA was injected into zygotes from C57 mouse (Beijing Vital River Laboratory Animal Technology Co.). Those fertilized eggs carrying mutants were then cultured until 2-cell embryos in M16 medium (Sigma, M7292) containing 10 nM PF477736 (Selleck, S2904), transplanted into pseudo-pregnant ICR female mice together with control and WT 2-cell embryos respectively, and then the litter sizes and body weight of each group were observed. All the experimental schemes of mice were reviewed and approved by the Institutional Review Board (IRB) of Reproductive Medicine, Shandong University.

### Copy Number Variant (CNV) Analysis

Whole genome amplification was performed, according to manufacturer's instructions, using the SurePlex WGA (VeriSeq PGS Kit, Illumina). The high-throughput sequencing platform, DA8600, was used for sequencing. CNV analysis was done by aligning the sequence of mutant blastocysts treated by PF477736 with the sequence of normal control blastocysts to detect if there are chromosome aneuploidy abnormalities or chromosomal deletions or duplications larger than 4 Mb. For ESCs derived from embryos treated with PF477736, WGA was performed with the same method using one outgrowth of the cell lines, following by library preparation and sequencing on the Miseq system (Illumina). CNV-seq results of mouse blastocysts with mutations after treatment with PF477736. The following Table shows the results of CNV analysis of mouse blastocysts with mutations after treatment with PF477736.

TABLE 8

| Blastocyst | CNV-seq result |
|---|---|
| F441fs*16-1 | Balanced, Euploid |
| F441fs*16-2 | Balanced, Euploid |
| F441fs*16-3 | Balanced, Euploid |
| F441fs*16-4 | Balanced, Euploid |
| R379Q-1 | Balanced, Euploid |
| R379Q-2 | Balanced, Euploid |
| R379Q-3 | Balanced, Euploid |
| R379Q-4 | Balanced, Euploid |

### Statistics Analysis

GraphPad Prism 8.0 was used for statistical analysis. Most experiments were repeated at least three times. Unpaired t-test or chi-square test was used for the comparison between two groups. The significant evaluation style of GraphPad is as follows: **P<0.01, ***P<0.001, ****P<0.0001.

### Embryonic Development Culture Additive

After determining that mouse zygotes overexpressing wild-type CHK1 could normally develop to blastocysts, Applicants also noted the cleavage rate and blastocyst development rate were significantly improved after adding CHK1 inhibitor. Based on this, further testing was performed to confirm whether a modified culture medium (i.e., a medium modified according to the general inventive concepts) could enhance blastocyst development rate.

CHK1 inhibitors were tested in three concentration ranges low concentration less than 0.1 nM (Low), medium concentration 0.1 nM to 100 nM (Medium) and high concentration greater than 100 nM (High)(concentration levels based on total medium volume).

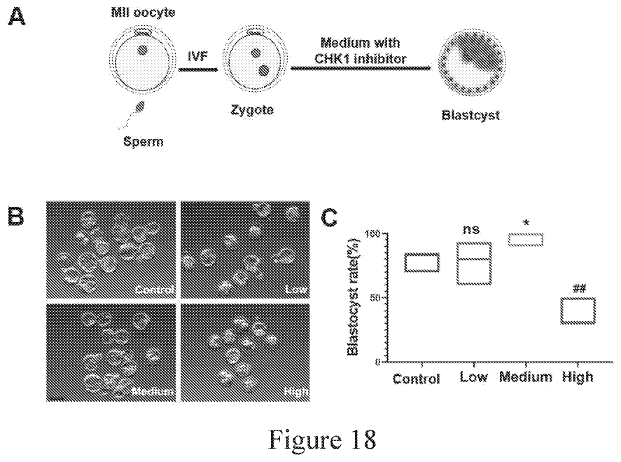

In general, 4-6 hours after fertilization, the fertilized eggs with obvious double pronuclei were obtained and added to the medium supplemented with different doses of CHK1 inhibitor, and cultured to the blastocyst stage (FIG. 18A), and the proportion of blastocyst development among different groups (development rate and embryo quality) was compared. The results showed that adding a certain dose of CHK1 inhibitor (e.g., a medium dose) could significantly increase the blastocyst development rate. In contrast, the low-dose group had no significant benefit in increasing the blastocyst development rate, while the high-dose group inhibited embryonic development (e.g., quality). Accordingly, the general inventive concepts recognize that particular dose range of CHK1 inhibitor did significantly promote blastocyst development (FIGS. 18 B&C).

Thereby, the general inventive concepts are also based, in part, on the discovery that embryonic development can be enhanced or improved by the addition of an additive to the culture medium. The improved medium can improve the early embryonic development of mammalian embryos, including promoting blastocyst development rate and avoid embryonic quality issues associated with other therapies. In certain embodiments, the additive is selected from a CHK1 inhibitor. In certain embodiments, the additive is selected from a first-generation inhibitor e.g., PF477736 and/or AZD7762, etc., second-generation inhibitor CCT245737, etc.

The general inventive concepts also recognize and relate to the process of producing a culture medium and culturing a mammalian embryo in said culture. As shown herein, the additive can significantly improve blastocyst development rate in conventional mammalian embryo medium and various commercial medium without affecting embryonic developmental potential, and can be combined with various additives known to significantly promote early embryo development.

### Blastocyst Development Culture Methods

In vitro fertilization of mouse oocytes: Appropriate age female mice were selected for superovulation: intraperitoneally inject 5 IU pregnant horse serum gonadotropin (PMSG), 44-48 hours later inject 5 IU human chorionic gonadotropin (HCG). Cumulus oocyte complex (COC) were collected in the ampulla of the fallopian tube after about 16 hours. Sperm were collected from the cauda epididymis of male mouse and capacitated in conventional capacitation medium for 1 hour. COC and capacitated sperm were added to conventional in vitro fertilization medium covered with mineral oil, and cultured at 37° C. and 5% CO₂. After 4-6 hours, the formed zygotes were transferred into embryo medium covered with mineral oil and cultured continuously at 37° C. under 5% CO₂ conditions until blastocyst stage.

### Preparation of Embryo Development Medium Comprising an Additive (e.g., CHK1 Inhibitor)

Those of ordinary skill in the art will recognize the applicability of the instant additives of the present invention can confer to any suitable mammalian embryo culture medium known in the art, including, for example, bicarbonate buffered medium, Hepes buffered or MOPS buffered medium or phosphate buffered saline, examples of commonly used mediums include G1/G1-Plus, G2/G2-Plus, G-MOPS, KSOM, M16, M2, PBS. Further, in certain embodiments, the additive is used in conjunction with known supplementary factors that promote early embryo development, such as human serum albumin, fetal bovine serum albumin, growth hormone, melatonin, IGF2, etc. Such enhanced mediums can improve at least one aspect of embryonic development, including but not limited to the speed and quality of mammalian early embryo development.

### Culturing Embryo in the Embryo Medium Containing a CHK1 Inhibitor

The embryo culture medium (e.g., G1-plus) was equilibrated under suitable conditions for an appropriate time in advance, and the respective CHK1 inhibitor was added. The mouse oocytes were fertilized in vitro according to the method described above. After 4-6 hours, the formation of male and female pronuclei of the fertilized eggs could be observed. At this time, the fertilized eggs were transferred into embryo medium containing the CHK1 inhibitor and continue to culture until blastocyst stage, or change to embryo medium without inhibitor after embryo develops to late stage 2 cells and culture to blastocyst.

### Blastocyst Development

To test the quality of embryos obtained after adding CHK1 inhibitor, Applicants rated blastocysts in each concentration group according to the following criteria:
Grade 1: Early blastocyst with chamber, the blastocoel cavity is less than ½ of the embryo's volume.
Grade 2: The blastocoel volume is greater than or equal to ½ of the volume.
Grade 3: The blastocyst is fully expanded and the blastocoel cavity occupies the embryo.
Grade 4: The blastocyst is fully expanded, the blastocyst cavity is larger than the early embryo, and the zona pellucida is thinned.
Grade 5: The hatching blastocyst, the trophoblast begins to break through the transparent layer.
Grade 6: hatched blastocyst, the blastocyst hatched completely from the zona pellucida.

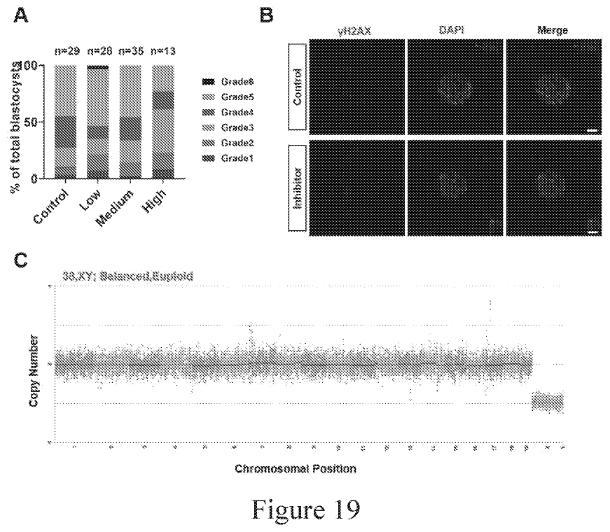

The results showed that the proportion of embryos of all grades, especially high-quality embryos (Grade 4/5/6) in the middle dose group was similar to that in the no inhibitor group (FIG. 19A). At the same time, the embryos obtained in the medium dose group were subjected to immunofluorescence staining of the DNA damage marker protein γH2AX, and the results showed that the blastocysts in the inhibitor group did not significantly increase the expression of γH2AX (FIG. 19B). Further, we selected blastocysts in the medium dose group for CNV—seq analysis, and the results showed that the embryos in the inhibitor group were ploidy intact, and there was no obvious deletion or duplication of large chromatin fragments (FIG. 19C). Taken together, the results demonstrate that adding an appropriate dose of a CHK1 inhibitor to the culture medium can significantly increase the blastocyst development rate without affecting the embryo quality.

As the general inventive concepts demonstrate, in certain embodiments (e.g., culture medium) the concentration of any additive, such as a CHK1 inhibitor, should be regulated to achieve the desired result (e.g., enhanced embryo development rate), while avoiding issues with embryo quality. In certain exemplary embodiments, the concentration of the additive (e.g., a CHK1 inhibitor) is 0.1 nM-100 nM.

The following Table is a list of CHK1 inhibitors contemplated for use in the compositions and methods according to the general inventive concepts.

TABLE 9

| Inhibitor | Companies | Clinical Trials | Countries | Notes |
|---|---|---|---|---|
| UCN-01 | | Phase 1, 2 | US; Canada | General I inhibitor |
| Prexasertib (LY2606368) | Array/Eli Lilly | Phase 1 | US | General I inhibitor |
| IC-83 (LY2603618) | Array/Eli Lilly | Phase 1, 2 | US | General I inhibitor |
| MK-8776 (SCH 900776) | Merck | Phase 1, 2 | US | General I inhibitor |
| AZD7762 | AstraZeneca | Phase 1 | US; Japan | General I inhibitor |
| CBP501 | CanBas | Phase 1, 2 | US | General I inhibitor |
| XL844 | Exelixis | Phase 1 | US | General I inhibitor |
| PF-477736 | Pfizer | Phase 1, 2 | US | General I inhibitor |
| SRA737 | Sierra Oncology | Phase 1, 2 | UK | General II inhibitor |
| GDC-0575 | Roche | Phase 1 | US | General II inhibitor |
| LY2880070 | Esperas/Eli Lilly | Phase 1, 2 | US; Canada | General II inhibitor |
| M4344 | Merck | Phase 1, 2 | US; Canada | |
| BEBT-260 | BeBetter Medicine | | | |
| CCT245737 | MedChemExpress | | | |
| CHIR-124 | MedChemExpress | | | |
| SAR-020106 | InvivoChem | | | |
| PD0166285 | MedChemExpress | | | |
| SB-218078 | MedChemExpress | | | |
| Chk1-IN-5 | MedChemExpress | | | |
| CCT244747 | MedChemExpress | | | |

Taken together, the results presented herein demonstrate that individuals having altered CHK1 function (including mutant CHK1 protein with increased kinase activity) in oocytes induces division failure of zygotes. Therefore, the general inventive concepts recognize the methods of inhibiting CHK1 kinase activity can successfully recover zygote division and accomplish the transition from meiosis to mitosis in early embryo development, thereby treating altered CHK1 activity and related infertility (e.g., ZA).

This is based in part on the discovery of novel dominant genetic mutations in CHK1 that cause female infertility induced by zygote arrest, characterized by pronuclear fusion failure. Applicants have also demonstrated that increased CHK1 activity caused by mutations arrests G2/M transition of zygotes. Importantly, administration of an inhibitor of CHK1 to suppress its kinase activity can rescue the zygote arrest phenotype in both mouse and human, offering an effective and safe treatment for this type of infertility. Applicants have also demonstrated that the addition of a CHK1 inhibitor to an embryonic culture medium can enhance the development rate of blastocytes while avoiding the previously known drawbacks related to embryo quality.

While various inventive aspects, concepts and features of the inventions may be described and illustrated herein as embodied in combination in the exemplary embodiments, these various aspects, concepts and features may be used in many alternative embodiments, either individually or in various combinations and sub-combinations thereof. Unless expressly excluded herein all such combinations and sub-combinations are intended to be within the scope of the present inventions. Still further, while various alternative embodiments as to the various aspects, concepts and features of the inventions—such as alternative materials, structures, configurations, and methods, such descriptions are not intended to be a complete or exhaustive list of available alternative embodiments, whether presently known or later developed. Those skilled in the art may readily adopt one or more of the inventive aspects, concepts or features into additional embodiments and uses within the scope of the present inventions even if such embodiments are not expressly disclosed herein. Additionally, even though some features, concepts or aspects of the inventions may be described herein as being a preferred arrangement or method, such description is not intended to suggest that such feature is required or necessary unless expressly so stated. Still further, exemplary or representative values and ranges may be included to assist in understanding the present disclosure, however, such values and ranges are not to be construed in a limiting sense and are intended to be critical values or ranges only if so expressly stated. Still further, exemplary or representative values and ranges may be included to assist in understanding the present disclosure, however, such values and ranges are not to be construed in a limiting sense and are intended to be critical values or ranges only if so expressly stated.

Parameters identified as “approximate” or “about” a specified value are intended to include both the specified value and values within 10% of the specified value, unless expressly stated otherwise. Further, it is to be understood that the drawings accompanying the present application may, but need not, be to scale, and therefore may be understood as teaching various ratios and proportions evident in the drawings. Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention, the inventions instead being set forth in the appended claims. Descriptions of exemplary methods or processes are not limited to inclusion of all steps as being required in all cases, nor is the order that the steps are presented to be construed as required or necessary unless expressly so stated.

1. A culture medium for mammalian embryo culturing, the culture medium comprising a therapeutically effective amount of a CHK1 inhibitor.

2. The culture medium of claim 1 comprising the CHK1 inhibitor in an amount of 0.1 nM to 100 nM.

3. The culture medium of claim 1, wherein the CHK1 inhibitor is at least one selected from Rabusertib, CCT245737, Prexasertib, AZD7762, and PF477736.

4. The culture medium of claim 1, wherein the CHK1 inhibitor is PF477736.

5. A method for enhancing embryonic development comprising: providing a culture medium, adding a therapeutically effective amount of a CHK1 inhibitor, and contacting the embryo with the culture medium.

6. The method of claim 5, wherein the CHK1 inhibitor is present in an amount of 0.1 nM to 100 nM.

7. The method of claim 5, wherein the CHK1 inhibitor is at least one selected from Rabusertib, CCT245737, Prexasertib, AZD7762, and PF477736.

8. The method of claim 5, wherein the CHK1 inhibitor is PF477736.

9. The method of claim 5, wherein development is enhanced by increasing blastocyst development rate.

10. A composition for reducing the risk of zygote arrest in an individual, the composition comprising a therapeutically effective amount of a CHK1 inhibitor.

11. The composition of claim 10, wherein the CHK1 inhibitor is PF477736.

12. A method for the treatment of infertility or altered kinase activity comprising, identifying an individual having altered CHK1 function, and administering therapeutically effective amount of a CHK1 inhibitor to the individual.

13. The method of claim 12, wherein the CHK1 inhibitor is at least one selected from Rabusertib, CCT245737, Prexasertib, AZD7762, and PF477736.

14. The method of claim 12, wherein the CHK1 inhibitor is PF477736.

15. A method for the treatment and/or prevention of zygote arrest, the method comprises identifying a subject suffering from zygote arrest or at increased risk of zygote arrest, contacting a zygote from the individual with a therapeutically effective amount of a CHK1 inhibitor.

16. The method of claim 15, wherein the CHK1 inhibitor is at least one selected from Rabusertib, CCT245737, Prexasertib, AZD7762, and PF477736.

17. The method of claim 15, wherein the CHK1 inhibitor is PF477736.

18. The method of claim 15, wherein the therapeutically effective amount of the CHK1 inhibitor is an amount sufficient to overcome the zygote arrest.

19. (canceled)

20. (canceled)

21. The method of claim 12, wherein the individual has increased kinase activity resulting in zygote arrest and/or infertility.


SEQUENCE LISTING (38 sequences)

SEQ ID NO: 1 - DNA, 43 bp, Artificial Sequence
  CHK1-LF097-Forward Primer
ggttggtcaaaagaatgacacaattctttaccaaattggatgc

SEQ ID NO: 2 - DNA, 43 bp, Artificial Sequence
  CHK1-LF097-Reverse Primer
gcatccaatttggtaaagaattgtgtcattcttttgaccaacc

SEQ ID NO: 3 - DNA, 49 bp, Artificial Sequence
  CHK1-LF057- Forward Primer
agaaatggatgataaaatattggttgacttcggctttctaaggtatttt

SEQ ID NO: 4 - DNA, 49 bp, Artificial Sequence
  CHK1-LF057- Reverse Primer
aaaataccttagaaagccgaagtcaaccaatattttatcatccatttct

SEQ ID NO: 5 - DNA, 53 bp, Artificial Sequence
  CHK1-LF033- Forward Primer
ggatgataaaatattggttgacttccagctttctaaggtatttttatgtttta

SEQ ID NO: 6 - DNA, 53 bp, Artificial Sequence
  CHK1-LF033- Reverse Primer
taaaacataaaaataccttagaaagctggaagtcaaccaatattttatcatcc

SEQ ID NO: 7 - DNA, 53 bp, Artificial Sequence
  CHK1-LF160- Forward Primer
ggttactatatcaacaactgataggaaaaacaataaactcattttcaaagtga

SEQ ID NO: 8 - DNA, 53 bp, Artificial Sequence
  CHK1-LF160- Reverse Primer
tcactttgaaaatgagtttattgtttttcctatcagttgttgatatagtaacc

SEQ ID NO: 9 - DNA, 31 bp, Artificial Sequence
  CDC25C- Forward Primer
agttctctggcatcgccggggagcgatatag

SEQ ID NO: 10 - DNA, 31 bp, Artificial Sequence
  CDC25C- Reverse Primer
ctatatcgctccccggcgatgccagagaact

SEQ ID NO: 11 - DNA, 58 bp, Artificial Sequence
  CDK1- Forward Primer
cccttatacacaactccagcggcaccttctccaattttctctattttggtataatctt

SEQ ID NO: 12 - DNA, 58 bp, Artificial Sequence
  CDK1- Reverse Primer
aagattataccaaaatagagaaaattggagaaggtgccgctggagttgtgtataaggg

SEQ ID NO: 13 - DNA, 20 bp, Artificial Sequence
  CHK1-RT- Forward Primer
gatcatcagcaatgcctcct

SEQ ID NO: 14 - DNA, 20 bp, Artificial Sequence
  CHK1-RT- Reverse Primer
ttcagctcagggatgacctt

SEQ ID NO: 15 - DNA, 20 bp, Artificial Sequence
  GAPDH-RT- Forward Primer
ctgcaccaccaactgcttag

SEQ ID NO: 16 - DNA, 20 bp, Artificial Sequence
  GAPDH-RT- Reverse Primer
ggatgcagggatgatgttct

SEQ ID NO: 17 - DNA, 20 bp, Artificial Sequence
  CHK1-Exon2- Forward Primer
gctgttaattttcgtgggca

SEQ ID NO: 18 - DNA, 20 bp, Artificial Sequence
  CHK1-Exon2- Reverse Primer
ttcagttgccaaaacccttg

SEQ ID NO: 19 - DNA, 22 bp, Artificial Sequence
  CHK1-Exon3- Forward Primer
tgagaacatagcagaaaccact

SEQ ID NO: 20 - DNA, 22 bp, Artificial Sequence
  CHK1-Exon3- Reverse Primer
tccaatttcacagttgcatgag

SEQ ID NO: 21 - DNA, 21 bp, Artificial Sequence
  CHK1-Exon4_5- Forward Primer
aagccccatatgtgttagtgg

SEQ ID NO: 22 - DNA, 23 bp, Artificial Sequence
  CHK1-Exon4_5- Reverse Primer
agacttgattttgccttgtatgg

SEQ ID NO: 23 - DNA, 20 bp, Artificial Sequence
  CHK1-Exon6- Forward Primer
tgatgaggggccttgcttta

SEQ ID NO: 24 - DNA, 19 bp, Artificial Sequence
  CHK1-Exon6- Reverse Primer
tctggccaagagtgagacc

SEQ ID NO: 25 - DNA, 22 bp, Artificial Sequence
  CHK1-Exon7- Forward Primer
tgaagtgcctctaaagtttcca

SEQ ID NO: 26 - DNA, 22 bp, Artificial Sequence
  CHK1-Exon7- Reverse Primer
tgctctgaatatacactcccca

SEQ ID NO: 27 - DNA, 21 bp, Artificial Sequence
  CHK1-Exon8- Forward Primer
actccaagatacagcagcaga

SEQ ID NO: 28 - DNA, 23 bp, Artificial Sequence
  CHK1-Exon8- Reverse Primer
gctatcatgtgttgttgacttgt

SEQ ID NO: 29 - DNA, 22 bp, Artificial Sequence
  CHK1-Exon9- Forward Primer
actccacactttgaacatgtct

SEQ ID NO: 30 - DNA, 21 bp, Artificial Sequence
  CHK1-Exon9- Reverse Primer
tcacacacaagttctcatgct

SEQ ID NO: 31 - DNA, 20 bp, Artificial Sequence
  CHK1-Exon10- Forward Primer
tcaggtggtgtgtcagagtc

SEQ ID NO: 32 - DNA, 20 bp, Artificial Sequence
  CHK1-Exon10- Reverse Primer
gcctccctcctctctttctt

SEQ ID NO: 33 - DNA, 20 bp, Artificial Sequence
  CHK1-Exon11- Forward Primer
gggaggccttcatgcaaaat

SEQ ID NO: 34 - DNA, 18 bp, Artificial Sequence
  CHK1-Exon11- Reverse Primer
caccccagcctcccaaaa

SEQ ID NO: 35 - DNA, 19 bp, Artificial Sequence
  CHK1-Exon12- Forward Primer
cctggtctgaagcgatcct

SEQ ID NO: 36 - DNA, 23 bp, Artificial Sequence
  CHK1-Exon12- Reverse Primer
agtctcttgtattgtcacccaga

SEQ ID NO: 37 - DNA, 20 bp, Artificial Sequence
  CHK1-Exon13- Forward Primer
aggaacagtgatgggcatga

SEQ ID NO: 38 - DNA, 23 bp, Artificial Sequence
  CHK1-Exon13- Reverse Primer
tggataaacagggaagtgaacac